(19) United States
(12) Patent Application Publication
(10) Pub. No.: US 2008/0257358 A1
(43) Pub. Date: Oct. 23, 2008

(54) Passive Treatment Device

(75) Inventors: Thomas P. Stern, Charlotte, NC (US); Edward S. Alessandrini, Fort Mill, SC (US); Lyndell D. Duvall, Fleetwood, NC (US); Anthony D. Pierce, Fleetwood, NC (US)

Correspondence Address: GROSSMAN, TUCKER, PERREAULT & PFLEGER, PLLC, 55 SOUTH COMMERICAL STREET, MANCHESTER, NH, 03101, US

(73) Assignee: GOODHEALTH, LLC, atlanta, GA (US)

(21) Appl. No.: 12/108,129

(22) Filed: Apr. 23, 2008

Related U.S. Application Data
(60) Provisional application No. 60/913,409, filed on Apr. 23, 2007.

(51) Int. Cl. A61M 16/00

(52) U.S. Cl. 128/207.16; 128/200.24

(57) ABSTRACT

The present disclosure relates to a device, system and method for treating sleep conditions that may include positioning into the mouth of a patient a device including a lower mouthpiece, including a locating device; an upper mouthpiece, including a housing; a valve body, insertable into the housing, including a channel having a slot defined therein for adjustably receiving the locating device in the slot, and at least one passageway; a first valve portion, insertable into the at least one passageway, including a slider; and a port, including a slide guide configured to slide-ably receive the slider.

### CROSS-REFERENCE TO RELATED APPLICATIONS

The present application claims the benefit of U.S. provisional patent application number 60/913,409 filed on Apr. 23, 2007, the disclosure of which is incorporated by reference herein.

### FIELD

The present disclosure relates to the treatment of conditions that may be caused by increased resistance or at least a partial occlusion of airways, which may include conditions such as snoring or sleep apnea. More specifically, the present disclosure relates to a device for the regulation or pressurization of exhaled air and maintaining airway integrity.

### BACKGROUND

Snoring and other sleep conditions such as apnea or hypopnea may commonly be caused by increased resistance and/or at least a partial occlusions in a person's airway. Increased resistance and obstruction may cause the oxygen levels in a person's blood to decrease and carbon dioxide levels to increase. In addition, increased resistance and/or at least a partial obstruction will cause sleep disruption. It is believed that these occlusions may be caused by conditions such as defects in the nasal septum, obesity, use of sedatives, alcohol or drugs, neuromuscular disease, weak respiratory muscles, collapse of the soft wall tissue in the airways, enlarged glands or nodes in the throat, etc. Current treatments may include the use of CPAP, APAP or VPAP machines; however, these machines require the use of an air compressor or other device to supply airway pressure.

### SUMMARY

An aspect of the present disclosure relates to a device for treating sleep apnea and/or other sleep conditions. The device may include a lower mouthpiece, including a locating device, an upper mouthpiece, including a housing, and a valve body, insertable into the housing. The valve body may include a channel having a slot defined therein for receiving the locating device in the slot and at least one passageway. In addition, the device may also include a first valve portion including a slider, wherein the valve is insertable into the at least one passageway, and a port, including a slide guide configured to slide-ably receive the slider.

Another aspect of the present disclosure relates to a system including the above device in combination with a nose piece. The nose piece may include, for example, a nose clip or a mask.

A further aspect of the present disclosure relates to a method of treating sleep apnea or other sleep conditions. The method may include positioning into a mouth a device including a lower mouthpiece, wherein the lower mouthpiece may include a locating device; an upper mouthpiece, wherein the upper mouthpiece may include a housing; a valve body, insertable into said housing, wherein the valve body may include a channel having a slot defined therein for adjustably receiving said locating device in said slot, and at least one passageway. The device may also include a first valve portion, insertable into the at least one passageway, wherein the first valve portion may include a slider and a port, including a slide guide configured to slide-ably receive the slider.

### BRIEF DESCRIPTION OF DRAWINGS

The features and advantages of this invention, and the manner of attaining them, will become more apparent and the invention will be better understood by reference to the following description of embodiments of the invention taken in conjunction with the accompanying drawings, wherein:

FIG. 1 is an illustration of the human respiratory system;

FIG. 2 is an illustration of pleural pressure over the course of the respiratory cycle;

FIG. 3 is an illustration of an example of a device for pressurizing airways;

FIG. 4 is another illustration of an example of a device for pressurizing airways;

FIG. 5 is an illustration of a connector for connecting valves to a port;

FIG. 6 is another illustration of an example of a device for pressurizing airways;

FIG. 7 a rear perspective view of an example of a device contemplated herein;

FIG. 8a is a rear perspective view of a lower mouthpiece of an example of a device contemplated herein;

FIG. 8b is a rear view of the lower mouthpiece of FIG. 8a;

FIG. 8c is a bottom view of the lower mouthpiece of FIG. 8a;

FIG. 9a is a rear perspective view of an example of an upper mouthpiece and housing contemplated herein;

FIG. 9b is a rear view of the upper mouthpiece and housing of FIG. 9a;

FIG. 9c is cross-sectional view A-A of the upper mouthpiece and housing illustrated in FIG. 9b;

FIG. 9d is a cross-sectional view B-B of the upper mouthpiece and housing illustrated in FIG. 9b;

FIG. 9e is a bottom view of the upper mouthpiece and housing of FIG. 9a;

FIG. 10a is a rear perspective of an example of a valve body contemplated herein;

FIG. 10b is a rear view of the valve body of FIG. 10a;

FIG. 10c is a cross-sectional view A-A of the valve body of FIG. 10b;

FIG. 10d is a cross-sectional view B-B of the valve body of FIG. 10b;

FIG. 10e is a rear view of the valve body of FIG. 10a;

FIG. 10f is a bottom view of the valve body of FIG. 10a;

FIG. 11a is a rear perspective view of an example of a first valve portion contemplated herein;

FIG. 11b is a side view of the valve portion of FIG. 11a;

FIG. 12a is a rear perspective view of an example of a second valve portion;

FIG. 12b is a side view of the second valve portion of FIG. 12a;

FIG. 12c is a top view of the second valve portion of FIG. 12a;

FIG. 13a illustrates a rear perspective view of a port contemplated herein;

FIG. 13b is a rear view of the port of FIG. 13a;

FIG. 13c is a front view of the port of FIG. 13a; and

FIG. 14 is an illustration of pleural pressure over the course of the respiratory cycle.

### DETAILED DESCRIPTION

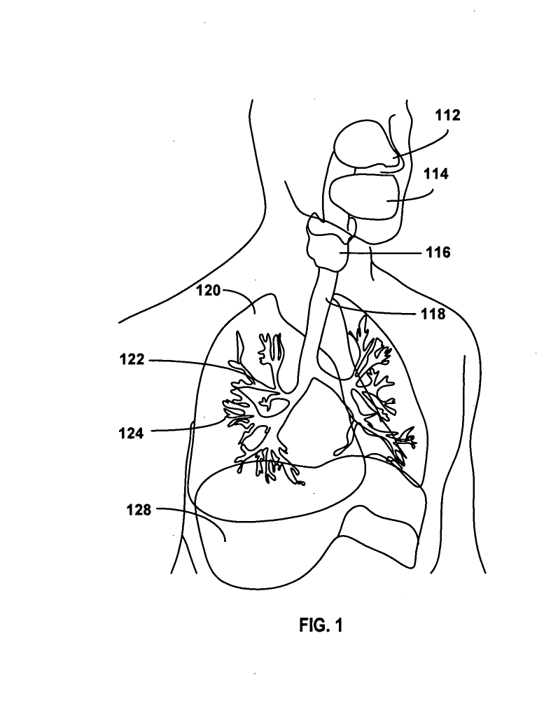

The present device relates to a mask and/or a mouthpiece device that pressurizes exhaled air to increase the pressure in the airway of a person during the rest portion of the respiratory cycle and maintaining the airway at least partially dilated through the entire respiratory cycle. An airway may be understood as those parts of the respiratory system through which air may flow. Accordingly, as illustrated in FIG. 1 an airway may therefore include the nose 112 and/or mouth 114, pharynx 116, and trachea 118, which lead to the lungs 120 including the bronchi 122, bronchioles 124, alveoli (not numbered), etc. In the present description, the term airway may also include any volume created within the device contemplated herein. It should also be understood that affixed to the chest wall and the lungs are the pleurae or membranes. The pleurae form a cavity called the pleural space.

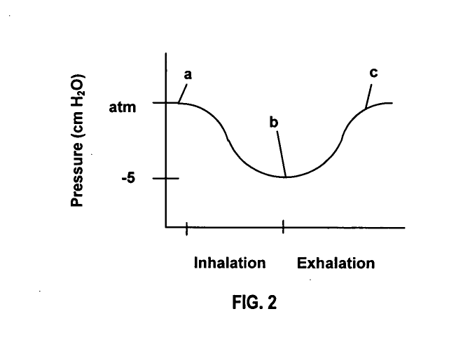

FIG. 2 illustrates a typical respiration cycle with respect to respiratory pressure versus time. When the respiratory system is at rest at the end of exhalation at point a, the pleural pressure and the pressure in all of the airways is equal to the pressure at the mouth, or atmospheric pressure. To move air into the lungs (120 illustrated in FIG. 1), the diaphragm (128 illustrated in FIG. 1) creates a pressure in the pleural space that is negative relative to the pressure at the mouth. During normal inspiration, between points a and b in FIG. 2, the diaphragm may create a negative pleural pressure of around −5 cm H₂O at point b. When the diaphragm relaxes, there is a pressure difference between the pleural space and the pressure at the mouth. The elasticity of the lungs allow for expiration at point as the lungs return to their pre-inspiration volume. The lungs contract, between points b and c, until the pressure in the pleural space reaches equilibrium again with the pressure at the mouth, which is in most cases atmospheric pressure, at point c. At this point, the respiratory system is once again at rest.

During the respiratory cycle, increased airway resistance or partial airway collapse may occur between the posterior end of the nasal septum and/or the epiglottis. Airway resistance and/or partial occlusion may be influenced by forces that may promote the collapse of the airway. Pressure which may promote at least a partial collapse may include pressure exerted on the airway by soft tissues and negative airway pressure created by the diaphragm. On the other hand however, airway resistance, or at least partial occlusion may be counteracted by forces that may cause dilation in the airway. Such dilation forces may include the action of pharyngeal dilator muscles and/or longitudinal traction of the airway from lung inflation. When forces that promote collapse of the airway overcome those forces that may otherwise dilate the airway, resistance or at least a partial occlusion of the airway may occur.

Accordingly, the device described herein may be utilized to increase pressure in the airway during the periods of exhalation and rest during the respiratory cycle, which may provide further dilation forces. The device may enclose the mouth and/or nose such that air may be substantially prevented from exiting the interface between the device and the mouth and/or nose area, allowing for pressure to develop between the device and the airway of a patient in the range of 0.1 to 30 cm H₂O, including all values and increments therein. The device may include one or more ports into which one or more valves may be affixed or formed integral to. Such valves may provide uni-directional or bi-directional flow to accommodate for inhaled and exhaled air.

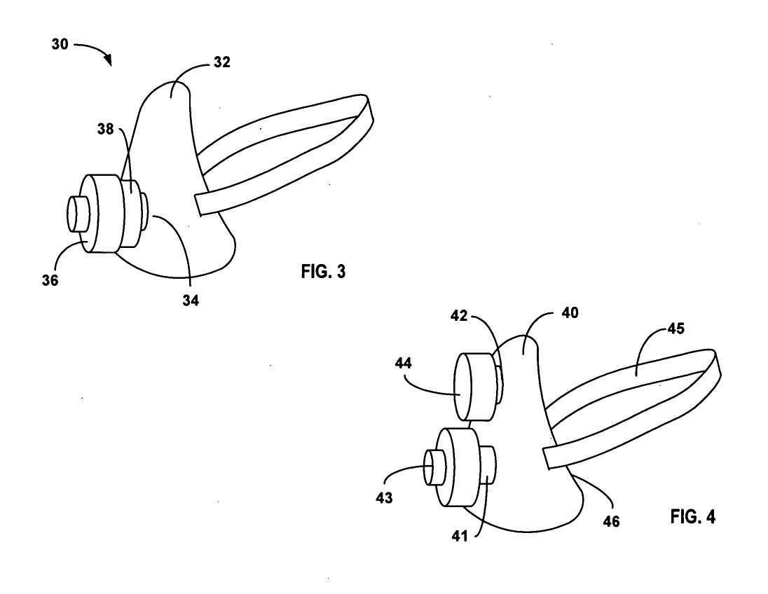

An exemplary device is illustrated in FIG. 3. This device 30 may include a mask 32 for covering both the mouth and nose which provides at least one port 34. A valve 36 to regulate air that is exhaled and a valve 38 to provide air to be inhaled may be connected to the port 34. The connection may be a removable connection or the connection may be an integral connection, i.e., the valves may be integrally formed into the device.

The valve for regulating exhalation may be a relief valve, which may be adjustable and set to a desired pressure such that upon meeting or exceeding such pressure, exhaled air may be released from the valve. The valve may include, for example, a PEEP valve or a spring loaded check valve. The valve may be used to generate positive end-expiratory pressure in the range of about 1 to 30 cm H₂O, including all values and increments therein. As noted above, the valve may rely on the use of a spring or the valve may rely on a resilient material to pressurize the airway. Accordingly, a desired airway pressure may be developed and may be maintained during the exhalation and rest portions of the respiratory cycle.

It should therefore be appreciated, that the pressure created in the airway during exhalation due to the presence of the pressure relief valve may remain above atmospheric pressure once the respiratory cycle is completed. Such pressure may promote the patency of the airway and help overcome those forces that may promote airway collapse. In addition, due to the increased pressure in the airways, the amount of air left in the lungs at the end of respiration, known as functional residual capacity (FRC), may be greater than without the device. This may lead to increased longitudinal traction of the airways, which may also promoting airway patency.

The inhalation valve, to provide air into the airway, may be a one-way valve, such as a check valve. Upon inhalation, the one way valve may open and then upon exhalation, the valve may close. The valve may assume a number of configurations and may be formed from a flap, membrane, disc or a duckbill valve. The valve may have a resistance to flow in the range of about 0.1 to 1.0 cm H₂O at a flow rate of 2 L/min, including all values and increments therein.

A further exemplary aspect of the device is illustrated in FIG. 4. In such an embodiment the mask 40 may include two ports 41 and 42 on which an exhalation valve 43 and an inhalation valve 44 are positioned, respectively. It should therefore be appreciated that the ports may be arranged in any manner or geometry. Accordingly, which the ports are illustrated as being arranged vertically, the ports may also be arranged horizontally or to either side of the nose and/or mouth.

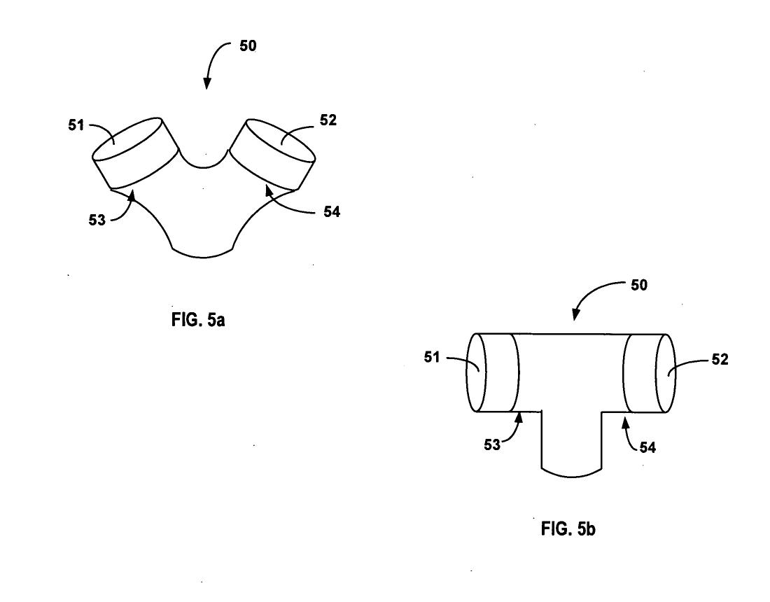

In addition, in another aspect other valve types may be utilized in the device. Additional valves may, for example, prevent the back flow of exhaled air, such as a check valve, which may be incorporated inline between the exhalation regulating valve and the port. Or, additional valves may provide for access ports in case of an emergency or failure of other valves in the device. In other embodiments, at least one port may be configured to include a T or Y connector 50 as illustrated in FIGS. 5a and 5b wherein the valves 51 and 52 may be affixed to respective ends 53 and 54 of the connector 50.

Accordingly, as can be seen from the aspects described above, at least one flow port and at least one valve may be provided in the system to accommodate for inhaled and/or exhaled air. In another aspect, at least two ports may be provided to accommodate for inhaled and exhaled air. It should be appreciated that while the illustrations above provide a single valve for inhalation and a single valve for exhalation, more than one valve may be provided for each function. In addition, the individual valve functions, i.e., inhalation and exhalation, may be combined into a single valve structure. Accordingly, a valve may be understood herein as a body which includes mechanisms, such as springs, flaps, membranes, etc., to provide and/or regulate the passage of air or another gas through the a body.

In another aspect, a mask may be provided wherein only the mouth is covered. In such a manner, the nose may be retained in a closed position, such that air does not pass through the nose, utilizing a nose clip. Furthermore, the mask may only cover the nose and in such a situation, the mouth may be enclosed utilizing a mouthpiece or clamp.

Referring back to FIG. 4, the mask 40 may also include a retention device 45, such as a strap. As illustrated, the strap may extend around the back of the head; however, other strap configurations are contemplated herein. Such configurations may include straps which may extend both around and over the head or straps that may extend around the ears. In addition, a cushion or seal may be provided around the periphery of the mask 46.

The mask may be formed of a thermoplastic material, such as acrylic, polycarbonate, polystyrene, etc. The retention device may be formed from, for example, an elastomeric or rubber material. The seal or cushion may be formed from silicone, rubber, elastomeric material or a combination thereof.

The mask may also include or be used in combination with a mandibular advancement device. The mandibular advancement device may be a mouthpiece inserted into the mouth to hold the tongue away from the back of the airway or to hold the lower jaw slightly forward relative to its natural relaxed position. Accordingly, the mandibular advancement device may counteract the pressure of the soft tissues, promoting airway patency. The mouthpiece may be formed integrally into the mask or may be removably affixed to the mask.

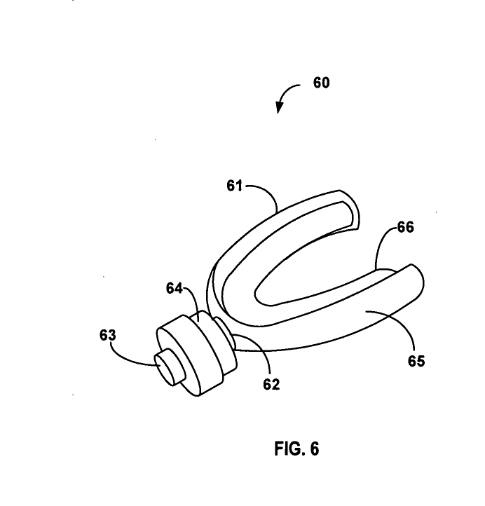

Another exemplary device is illustrated in FIG. 6. This device 60 may include a mouthpiece 61 which may be inserted and retained in the mouth. The mouthpiece may include a port 62, to which a valve 63 for regulation of exhaled air and a valve 64 to provide air for inhalation may be affixed. The mouthpiece 61 may also include a wall 65, which may be inserted between the teeth and gums. The mouthpiece 65 may also include a bridge 66, which may protrude between and may be contacted by at least a portion of the teeth.

The mouthpiece may also be used as a mandibular advancement device, as described above. Accordingly, in such a manner, the wall 65 and the bridge 66 of the mouthpiece may be formed to position the jaw in a slightly forward position relative to a natural relaxed position or the bridge may be replaced by retaining device capable of holding the tongue in a forward position, such that it does not fall back towards the airway.

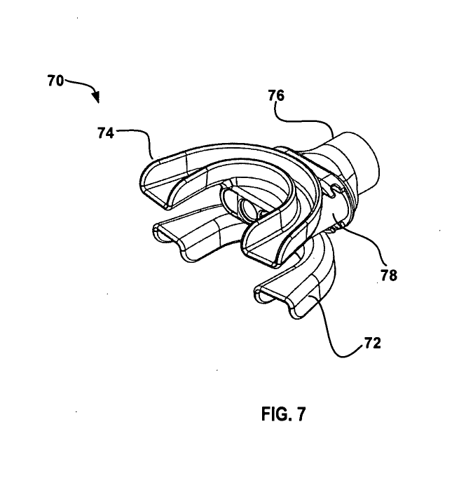

In a further example, illustrated in FIG. 7, and expanding on the examples above, a device 70 may be provided, wherein the device may include one or more mouthpieces 72 and 74, an inhale/exhale port 76 and a valve body 78. Each piece (i.e., the mouthpieces, port and valve body) may be provided as a separate portion and the device may be assembled together. In addition, the position of the mouthpieces relative to each other may be adjustable to accommodate individual patients.

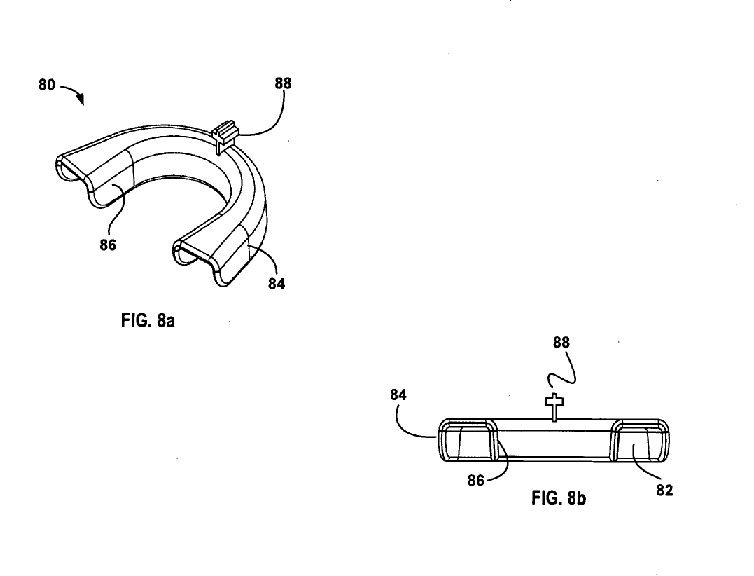

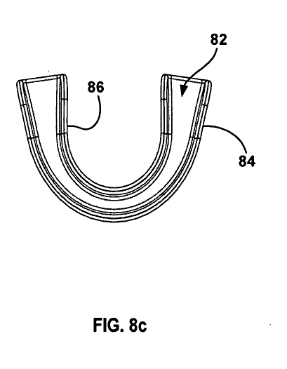

As illustrated in FIGS. 8a, 8b and 8c, an example of the lower mouthpiece 80 may be generally “U” shaped and define a cavity 82 so as to cover at least a portion of the teeth and/or gums. As illustrated, the sides of the mouthpiece 84 and 86 extend to about the same length; however one side may be shorter than the other. In addition, the lower mouthpiece may include a locating device 88, which may be used to attach the lower mouthpiece to the remainder of the device. In addition, the location of the lower mouthpiece may be adjustable with respect to the upper mouthpiece, as will be discussed further below, to provide mandibular advancement.

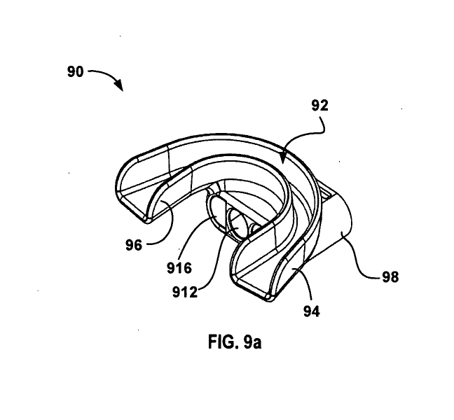

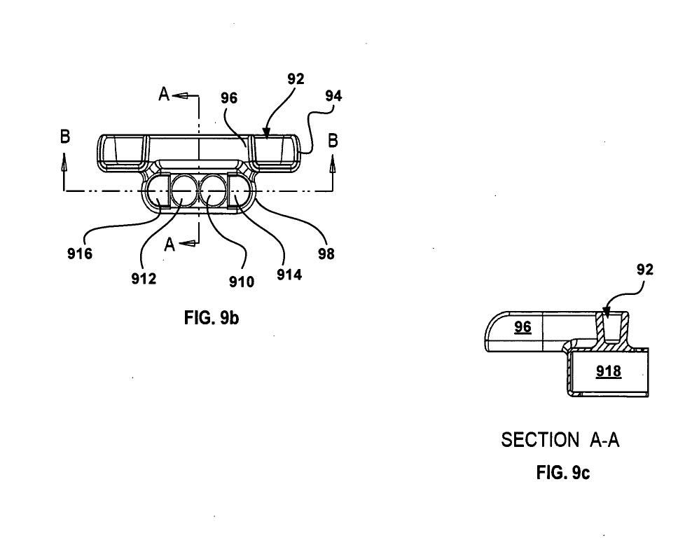

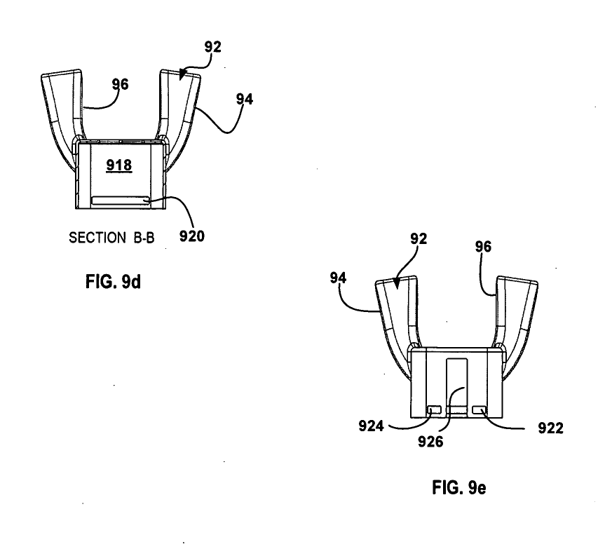

FIGS. 9a and 9b illustrate an example of an upper mouthpiece 90. Again, the upper mouthpiece 90 may be generally “U” shaped and define a cavity 92 for accommodating at least a portion of the upper teeth and/or gums. Once again, the sides of the mouthpiece 94 and 96 may extend to about the same length or may be different lengths. The upper mouthpiece 90 may also include a housing 98 for receiving at least a portion of the valve body (76 of FIG. 7). It may be appreciated that reference to upper and lower are simply a point of reference and that the device may be reversed, i.e., the lower portion may be placed proximate to the upper teeth and the upper portion may be placed proximate to the lower teeth.

FIG. 9c illustrates the housing taken at cross section A-A of FIG. 9b and FIG. 9d illustrates the housing taken at cross-section B-B of FIG. 9b. The housing may define a passageway 918 for receiving at least a portion of the valve body. In addition, the housing 98 may include at least one opening 920 for locating the valve body with respect to the housing. The opening defined by the housing may be in the shape of a slot, as illustrated which may receive a protrusion on the valve body, or other geometries may be defined as well, such as round, half round or quarter round holes, as well as square, rectangle, and other openings.

In addition, as illustrated in FIG. 9e, to further secure the valve body to the housing 98, the housing may include additional openings 922 and 924 defined in the housing for receiving protrusions from the valve body opposite the first opening 920. A further opening 926 may also be defined or provided in the valve body for receiving a slotted channel in the valve body, which may receive the locating device (88 illustrated in FIG. 8) of the lower mouth portion. In another example, the upper mouthpiece 90, itself, may include a slotted channel for receiving the locating device (88 illustrated in FIG. 8) of the lower mouth portion.

Referring back to FIG. 9b, the housing 98 may include one or more holes 910 and 912 for communicating air into and out of the valve body. In some examples, more than two holes may be present. The housing 98 may also include one or more valves 914 and 916 for communicating air into and out of the valve body. The valves may be formed into the housing and may be supported by the valve body. The valves may be in the form of flaps (as illustrated) or may be other valves, such as duck bill valves. Upon opening, the valves may allow air to pass through the valve body. When closed, the valves may seal against the housing and/or be supported by the valve body.

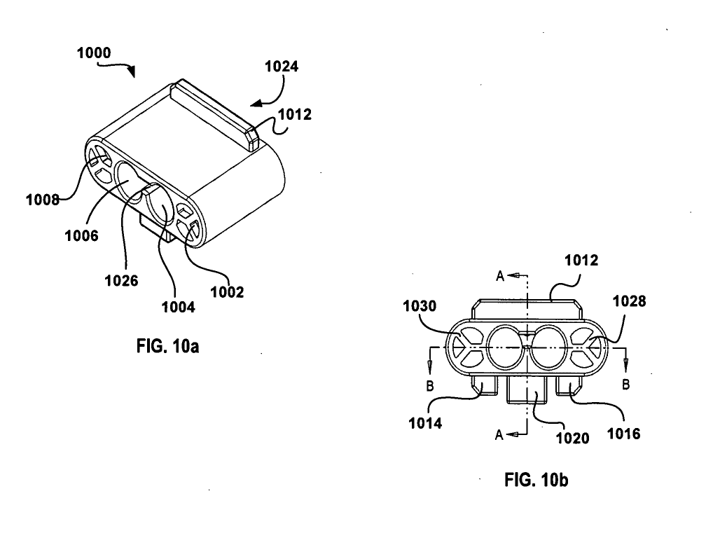

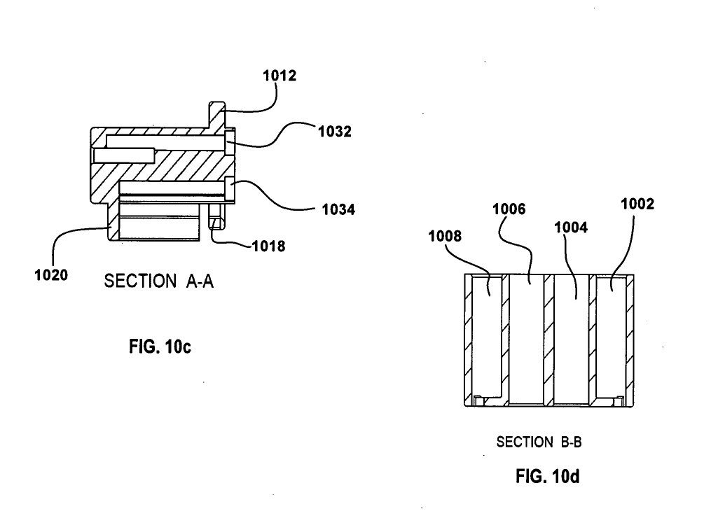

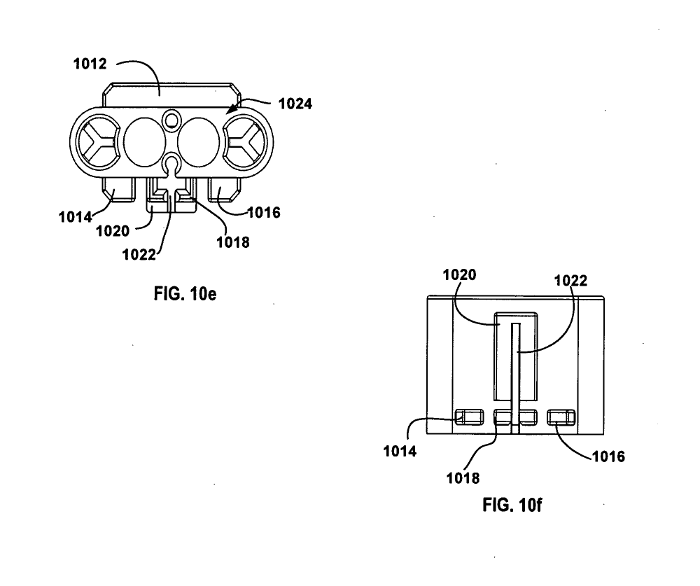

An example of a valve body is illustrated in FIGS. 10a, 10b, 10c, (which is a cross-section of 10b at A-A), 10d, (which is a cross-section of 10b at B-B), 10e and 10f. The valve assembly 1000 may include a number of ports or passageways 1002, 1004, 1006, 1008 for communicating air into and out of the airway of a patient. The passageways may be isolated or one or more of the passageways may be partially or completely open to one or more other passageways. Valves may be positioned in communication with the passageways to accommodate for air inhalation and exhalation.

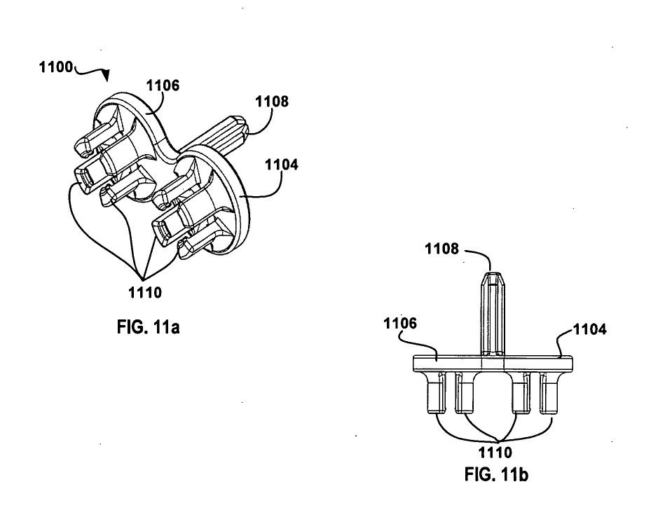

Illustrated in FIGS. 11a-b is an example of a first valve portion 1100 which may be positioned in passageways 1004 and 1006 on the distal end of the valve body (1024 of FIG. 10). Each disc shaped portion 1104 and 1106 forms a valve disc, joined in the center and including a distal stem or slider 1108. The discs may include a seating gasket in the form of a ring or disk that may seat against the valve body during operation. The seating gasket may be a natural rubber, silicone, synthetic rubber, thermoplastic elastomer, etc. The valve may also include magnets or springs retained by one or more fingers 1110. The valve 1100, and in particular the fingers 1110, may slide back and forth in the passageways along the slider 1108, which may be positionable in the port (76 of FIG. 1).

The first valve portion may be configured to unseat from the valve body upon the application of a given pressure developed in the airway upon exhalation. The pressure may be determined and adjusted for a given user. The valve may then remain open while the given pressure is exceeded and once the pressure falls below the given pressure, the valve may reseat. Reseating of the valve may occur due to gravity, i.e., the weight of the valve, or due to the assistance of a spring affixed to the valve body or a second valve portion.

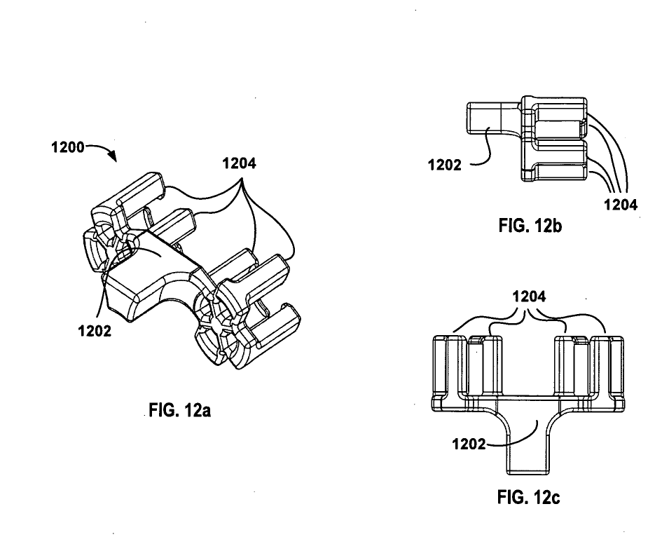

An example of a second valve portion 1200 is illustrated in FIGS. 12a, 12b, and 12c. The second valve portion may be retained by the valve body. For example, the second valve portion may include a bridge 1202 which may slide into channel 1026 (illustrated in FIG. 10) defined by the valve body. The second valve portion may also include magnets, which may be attracted to the magnets held by the first valve portion, or the second valve portion may receive the springs held by the first valve portion 1100. The second valve portion 1200 may also include one or more of fingers 1204 in which the magnets or spring may be retained. In one example, the fingers 1204 may slide with respect to the valve fingers 1110 (illustrated in FIG. 11) in an interlocking manner. In another example, both sets of fingers may include projections which may catch the projections of the other sets of fingers when fully extended. Air may pass through the second valve portion and develop pressure against the first valve portion.

When exhalation pressure is exerted on the first valve portion, and reaches and/or exceeds a given pressure, the magnets may separate or the spring may extend allowing for the first valve portion to unseat and slide forward and air to escape the valve body. After the given pressure is no longer exceeded, the first valve portion may then slide back to its seated position due to the forces exerted by the magnets, relaxation of the springs or gravity.

During inhalation, the first valve portion may remain seated and valve flaps 914 and 916 located in the housing 98 (illustrated in FIG. 9) may be drawn outwards, away from the valve body and/or towards the tongue. The flaps may be supported by support structures 1028 and 1030 (illustrated in FIG. 10) during inhalation. The flaps may also seal against the housing 98 (illustrated in FIG. 9). It is also envisioned that the flaps or other valves may be positioned within the valve body and may otherwise collapse during inhalation. For example, duck bill shaped valves formed of a flexible material, may allow air to pass into the device and may otherwise block air being exhaled.

Referring back to FIGS. 10a-f, as alluded to above, the valve body 1000 may also include one or more projections 1012, 1014, 1016, 1018 for positioning and/or retaining the valve body within the housing 98. The projections may fit within the openings 920, 922, 924 and 926 defined by the housing 98. The projections may extend from the exterior surface of the housing or may be flush with the housing.

Furthermore, the valve body may also include a channel 1020 including a slot 1022 (also alluded to above) for receiving the locating device of the lower mouthpiece (88, illustrated in FIG. 8). The locating device may be adjusted proximally or distally in the slot up to two centimeters, including all values and increments in the range of, for example, 0.01 to 2.0 cm. The slot defined within the channel and corresponding locating device may assume a number of geometries. As illustrated the slot defined in the channel is cross shaped, however, the slot may also be T-shaped, dovetail shaped, etc. The slot may also be defined in the projections 1018 as well, for receiving the locating device of the lower mouthpiece.

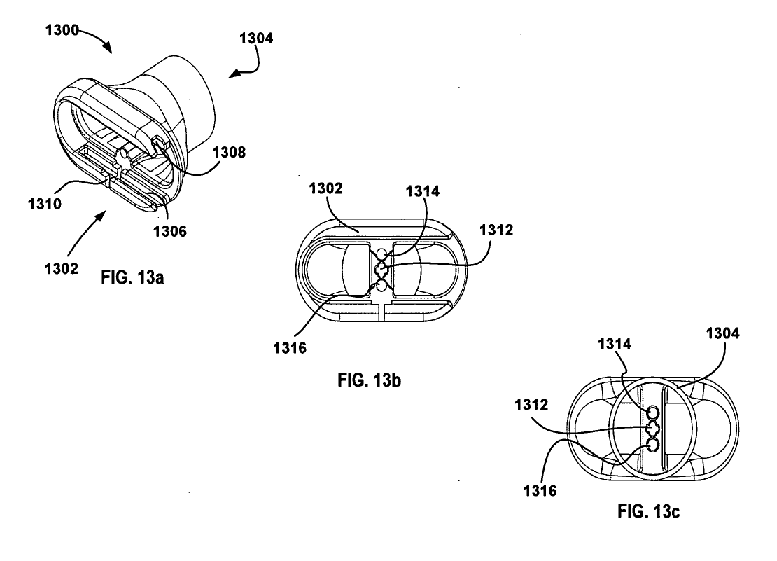

In addition, an intake/exhalation port may be provided as illustrated in FIGS. 13a, 13b, and 13c. The port 1300 may include a proximal end 1302 and a distal end 1304. The proximal end of the port may include one or more channels 1306 and 1308, which may receive the projections formed in the valve body and extending from the housing. The entrance of the channels may also include projections or ridges which may retain the valve body projections in the channels, preventing the port from disassociating with the valve body and/or housing. As the valve body projections (1012-1018 illustrated in FIGS. 10a-f) are passed over the channel ridges the port may deflect and then relax once the projections are seated in the channels 1306 and 1308. In addition, the proximal end of the port 1300 may include a slot 1310 defined therein for receiving or accommodating the locating device 88 (illustrated in FIG. 8) of the lower mouth portion. The port 1300 may also include a slide guide 1312 for guiding the slider 1108 of the first valve 1100 (illustrated in FIG. 11).

Furthermore, the port 1300 may include two locating screw holes or guides 1314 and 1316. Adjustment screws may be inserted into the screw holes or guides, extending into the valve body (as illustrated by 1032 and 1034 of FIG. 10c). In one example, one of the screws may engage the second valve portion 1200 (illustrated in FIG. 12), and may adjust the pressure required to unseat the exhalation valve, such as, for example, by adjusting the spring force. In another example, one of the screws may engage the locating device 88 for the lower mouthpiece (illustrated in FIG. 8), allowing for the adjustment of mandibular advancement. Threads or teeth may be provided in the bridge 1202 of the second valve portion (illustrated in FIG. 12) or the locating device 88 (illustrated in FIG. 8) for receiving the screws. The port 1300 may also include an indentation, not illustrated, in the exterior surface to accommodate the lips of a user. The port may be relatively low profile, and may extend just outside of the mouth and/or about 2 cm or less from the exterior surface of the mouth.

The mouthpieces described herein may be formed from a moldable plastic material. The material may be either a thermoplastic or a thermoset. For example, the mouthpiece may be formed from a thermoplastic material that may be boiled, causing the thermoplastic material to become shapeable and upon cooling capable of retaining a formed shaped. In addition, the mouthpiece may be formed from a thermoset material that may be cast into impressions of an individual's mouth/teeth. Furthermore, the material may be relatively flexible.

The valve body and/or valve portions may be formed of a thermoplastic material, such a polystyrene, nylon, acrylic, polycarbonate, etc. In addition, the valve body or valve portions may be formed of a relatively hard natural or synthetic rubber, silicone or a thermoplastic elastomer. In addition, the port may also be formed of similar materials.

During use the device may be positioned into the mouth of the patient and the position of the lower mouthpiece may be adjusted relative to the upper mouthpiece by moving the locating device in said slot to provide mandibular advancement. In addition, the device may be used in combination with a nose piece, such as a clip, plugs, or other device to prevent the inhalation or exhalation of air through the nose. Furthermore, the above may be integrated into a mask as described above.

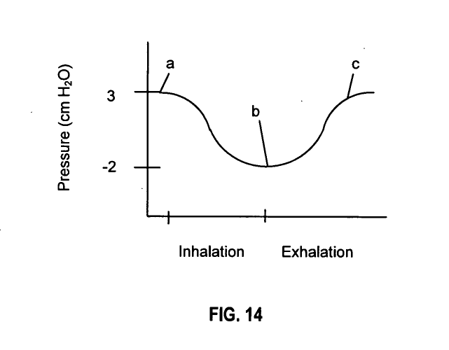

FIG. 14 illustrates an exemplary respiratory cycle for a person utilizing a device contemplated herein with respect to pressure versus time. When the respiratory system is at rest at the end of exhalation at point a, the pleural pressure and the pressure in all of the airways is substantially similar or less than the pressure set on the pressure relief valve in the device. For illustrative purposes, the setting is assumed to be 3 cm H₂O. To move air into the lungs (120 illustrated in FIG. 1), the diaphragm (128 illustrated in FIG. 1) creates a pressure in the pleural space that is negative relative to the pressure at the mouth and the pressure setting of the pressure relief valve of the device. During inspiration, between points a and b in FIG. 14, the diaphragm may create a negative pleural pressure of around −5 cm H₂O at point b drawing air though a check valve in the device. Therefore in the example above, this may decrease the pressure in the pleural space to a value of approximately −2 cm H₂O. When the diaphragm relaxes, the elasticity of the lungs allows for expiration at point b and the lungs may return to their pre-inspiration volume. The lungs may contract, between points b and c, until the pressure in the pleural space reaches equilibrium again with the pressure at the mouth, at or just below the pressure relief valve setting at point c, which in the illustrative example would raise the pressure back to around 3 cm H₂O. At this point, the respiratory system is once again at rest.

Accordingly, in a broad aspect, the combination of the mouthpiece and a pressure relief valve may be employed in a method to raise pleural pressure through out expiration and while the respiratory system is at rest. The positive pressure in the airway in combination with mandibular repositioning may be pressurize the airway during the rest portion of the respiratory cycle and maintain the airway at least partially dilated through the entire respiratory cycle.

A further aspect of this disclosure therefore relates to a system, device and/or method for treating sleep conditions that may include a device capable of enclosing the mouth and/or nose of a person having at least one port defined therein, including a relief valve and an inhalation valve. The system may also include a mandibular advancement device capable of being inserted into the mouth of said person.

Another aspect of this disclosure relates to a device for treating sleep conditions comprising a mask having at least one port defined therein, including a relief valve and an inhalation valve. The device may also have a mandibular advancement device affixed to the mask.

In addition, a further aspect of this disclosure relates to a device for treating sleep conditions including a mandibular advancement device capable of being inserted into the mouth of a person, having a at least one port defined therein, including a relief valve and an inhalation valve.

The foregoing description of several methods and an embodiment of the invention has been presented for purposes of illustration. It is not intended to be exhaustive or to limit the invention to the precise steps and/or forms disclosed, and obviously many modifications and variations are possible in light of the above teaching.

What is claimed is:

1. A device for treating sleep apnea comprising:
a lower mouthpiece, including a locating device;
an upper mouthpiece, including a housing;
a valve body, insertable into said housing, including
a channel having a slot defined therein for receiving said locating device in said slot, and
at least one passageway;
a first valve portion including a slider, wherein said first valve portion is insertable into said at least one passageway; and
a port, including a slide guide configured to slide-ably receive said slider.

2. The device of claim 1, wherein said valve body further comprises two or more passageways.

3. The device of claim 1, further comprising a second valve portion configured to release said first valve portion upon application of a given pressure.

4. The device of claim 3, wherein said first and second valve portions include magnets.

5. The device of claim 3, wherein said first valve portion includes a spring and said second valve portion retains said spring.

6. The device of claim 1, wherein said housing includes at least one third valve, wherein said at least one third valve, in a closed position, covers said passageway and in an open position allows passage of air through said passageway.

7. The device of claim 6, wherein said valve body supports said at least one third valve.

8. The device of claim 1, wherein said valve body includes projections and said housing includes openings for receiving said projections.

9. The device of claim 1, wherein the position of said locating device may be adjusted in said slot.

10. The device of claim 1, further comprising adjusting screws, wherein said port includes screw holes for receiving said adjusting screws.

11. The device of claim 10, wherein said valve body includes screw holes for receiving said adjusting screws.

12. The device of claim 10, wherein said adjusting screws are configured to adjust said lower mouthpiece with respect to said upper mouthpiece.

13. A system for providing sleep apnea treatment, comprising:
a device including:
a lower mouthpiece, including a locating device;
an upper mouthpiece, including a housing;
a valve body, insertable into said housing, including
a channel having a slot defined therein for adjustably receiving said locating device in said slot, and
at least one passageway;
a first valve, insertable into said at least one passageway, including a slider; and
a port, including a slide guide configured to slide-ably receive said slider; and
a nose piece.

14. The system of claim 13, wherein said nose piece includes a nose clip.

15. The system of claim 13, wherein said nose piece includes a mask and said device is integrated into said mask.

16. A method of treating sleep apnea, comprising:
positioning into a mouth a device including a lower mouthpiece, including a locating device; an upper mouthpiece, including a housing; a valve body, insertable into said housing, including a channel having a slot defined therein for adjustably receiving said locating device in said slot, and at least one passageway; a first valve portion, insertable into said at least one passageway, including a slider; and a port, including a slide guide configured to slide-ably receive said slider.

17. The method of claim 16, further comprising adjusting the position of said lower mouthpiece relative to said upper mouthpiece by moving said locating device in said slot.

18. The method of claim 16, further comprising unseating said first valve portion from said passageway, wherein said slider slides in said slide guide, upon the application of a given pressure.

19. The method of claim 16, further comprising providing a nose piece.

20. The method of claim 16, taking air into an airway through said at least one passageway and into said mouth; and exhaling air through said passageway, wherein said first valve portion pressurizes said air in said airway.
